(19) United States
(12) Patent Application Publication
(10) Pub. No.: US 2010/0129455 A1
(43) Pub. Date: May 27, 2010

(54) NANOPARTICLE-DISPERSED FINE GLASS BEADS HAVING A CAVITY THEREIN, AND METHOD OF PRODUCING THE SAME

(75) Inventors: Norio MURASE, Osaka (JP); Ping YANG, Osaka (JP); Masanori ANDO, Osaka (JP)

Correspondence Address: KRATZ, QUINTOS & HANSON, LLP, 1420 K Street, N.W., Suite 400, WASHINGTON, DC, 20005, US

(73) Assignee: NATIONAL INSTITUTE OF ADVANCED INDUSTRIAL SCIENCE AND TECHNOLOGY, Tokyo (JP)

(21) Appl. No.: 12/578,051

(22) Filed: Oct. 13, 2009

(30) Foreign Application Priority Data
Oct. 15, 2008 (JP) 2008-266176
May 14, 2009 (JP) 2009-117244

(51) Int. Cl. B32B 5/16; C03B 19/10; H01F 1/00; A61K 9/14; A61K 31/192; A61P 29/00

(52) U.S. Cl. 424/489; 428/402; 428/406; 65/21.4; 252/62.51 R; 514/576; 977/773

(57) ABSTRACT

The present invention provides fine silicon-containing glass beads each having one or more cavities therein and containing nanoparticles in a glass phase of each of the silicon-containing glass beads, and a method of producing such glass beads, and also provides silicon-containing glass beads containing nanoparticles, which may be identical to or different from the nanoparticles in the glass phase, and a functional material such as pharmaceutical molecules (e.g., materials having fluorescent properties, magnetic properties, drug effects, etc.), and a method of producing such glass beads.

The present invention relates to silicon-containing glass beads each having one or more cavities therein and having an average particle diameter of 20 nm to 1 μm, the silicon-containing glass beads containing nanoparticles A in the silicon-containing glass phase, and also containing a functional material in the cavity.

### TECHNICAL FIELD

The present invention relates to fine glass beads having a cavity therein and containing nanoparticles dispersed in the glass phase, and a method of producing such glass beads.

In the specification, a silicon-containing matrix produced by the sol-gel method is referred to as “silicon-containing glass”. The silicon-containing glass generally does not have a clear peak in an X-ray diffraction. Moreover, the present invention is aimed at fine (about 1 μm or less in diameter), nearly spherical silicon-containing glass particles, which are referred to as “silicon-containing glass beads”.

### BACKGROUND ART

Glass has superior chemical resistance and mechanical properties compared to polymers, as well as excellent ultraviolet (UV) resistance. Glass is therefore useful as a matrix for supporting nanoparticles and organic molecules having various functions.

A lot of research and development have been performed on fine beads in which glass is used as the matrix, and many methods of producing silicon-containing glass by the sol-gel method have been reported. In these methods, when glass beads are produced under alkaline conditions and under the condition where the temperature of heat treatment is low, the network structure of silicon oxide is insufficiently developed. In some cases, large amounts of organic substances may be contained, or aggregates of about 1 nm-diameter fine silica spheres may be formed.

One of such silicon-containing glass beads already-commercialized is “inorganic spherical fine particles” (trade name: Godd Ball) produced by Suzuki Yushi Industrial Co., Ltd. The glass beads are used as fillers for chromatography, cosmetic powder, etc. Depending on the production methods, a hollow structure can be formed near the center of the bead. Since glass beads having a hollow portion are able to support and slowly release a drug, they may be used in drug delivery. Many of such silicon-containing glass beads have a particle diameter of about 1 to 100 μm. Beads with a particle diameter of 1 μm or less make it difficult to control the particle diameter or hollow core diameter.

Moreover, PTL 1 discloses hollow particles composed of a silicon-containing shell, having macropores (60 nm to 30 μm in diameter) in the shell portion (about 0.2 to 100 μm in diameter) of the spherical particle, and a production method thereof. In relation to this, NPL 1 teaches a method of producing such hollow particles in detail.

On the other hand, studies on supporting nanoparticles in silicon-containing glass beads have also been progressing. Nanoparticles are particles of about 2 to 20 nm in diameter, which have a large surface area, and thus, without some care, may often be readily aggregated, resulting in the loss of various functions. For this reason, it is critical to support nanoparticles in an appropriate matrix.

Among such nanoparticles, semiconductor nanoparticles have been actively studied in recent years. Semiconductor nanoparticles are mainly composed of a compound semiconductor of Group II-VI or III-V and are produced by the solution method. Those that eliminate surface defects are known as new-type phosphors. Even among semiconductor nanoparticles made of the same material, due to the so-called quantum size effect, the band gap increases as the particle diameter decreases, and leads to light emission at shorter wavelengths. In addition to semiconductor nanoparticles, magnetic nanoparticles and metallic nanoparticles are also known.

PTL 2 describes a structure of silicon-containing glass beads of several tens of nanometers in diameter having a cavity therein and containing semiconductor nanoparticles in the cavity, and a production method thereof.

Moreover, the present inventors have compiled techniques relating to silicon-containing glass beads in which luminescent semiconductor nanoparticles are dispersed and their production methods, as shown in PTL 3 (reverse micelle method), PTL 4 (the Stoeber method), and PTL 5 (improved reverse micelle method). According to these techniques, plural semiconductor nanoparticles can be dispersed in glass beads of about 30 nm to 1 μm in diameter while preventing the deterioration of quantum efficiency. However, these techniques are all intended to disperse one type of nanoparticles in glass beads.

Glass beads with various properties can be obtained by incorporating not only one type of nanoparticles, but also several other types of nanoparticles and functional materials, such as pharmaceutical molecules, into the glass beads. NPL 2 aims to produce such multifunctional glass beads. NPL 2 teaches the production of glass beads exhibiting fluorescent and magnetic properties, in which semiconductor nanoparticles and magnetic nanoparticles are dispersed in one glass bead.

Although the method of producing the semiconductor nanoparticles used in these glass beads was based on previously published NPL 3, the photoluminescence (PL) efficiency of the semiconductor nanoparticles was nowhere mentioned in NPL 2. When the full-width at half-maximum (FWHM) of the emission reported in NPL 2 was measured, it was as wide as 65 nm in the case of the red emission. It is therefore determined that the size selection treatment described in NPL 3 was not performed in NPL 2. In this case, the PL efficiency is about 10%. Moreover, when the semiconductor nanoparticles were introduced into the glass beads together with magnetic particles, the FWHM increased and the baseline level of emission was greatly increased. It is thus confirmed that the PL efficiency was greatly decreased.

Generally, when luminescent nanoparticles are mixed with magnetic nanoparticles, the interaction between both nanoparticles reduces the PL efficiency. For example, iron oxide nanoparticles are positively charged, whereas, many of water-dispersible semiconductor nanoparticles are negatively charged. Accordingly, aggregates are formed readily after mixing both nanoparticles so as to disperse them simultaneously in glass beads.

Moreover, the dispersion of nanoparticles is generally maintained by a delicate balance between the nanoparticles and the surrounding ions in the solution. Therefore, when other chemical species are added, the balance may often be disrupted, followed by aggregation of nanoparticles or degradation of the nanoparticle surface. This is efficiently prevented by separately dispersing nanoparticles and magnetic particles, pharmaceutical molecules, etc., in different areas of each fine glass bead. However, such a method has not been known.

### [Citation List]

### [Patent Literature]

### [PTL 1] Japanese Unexamined Patent Publication No. 2007-230794

### [PTL 2] Japanese Unexamined Patent Publication No. 2007-105873

### [PTL 3] Japanese Patent No. 3677538

### [PTL 4] Japanese Patent No. 3755033

[PTL 5] International Publication No. WO 2007/034877

### [Non-Patent Literature]

### [NPL 1] Fujiwara et al., Chemistry of Materials, vol. 16, p. 5420 (2004)

### [NPL 2] Veronica Salgueirino-Maceira et al., Advanced Functional Materials, vol. 16, p. 509 (2006)

### [NPL 3] Gaponik et al., Journal of Physical Chemistry B, vol. 106, p. 7177 (2002)

### SUMMARY OF INVENTION

### Technical Problem

The present invention was achieved in view of the current state of the foregoing prior art. A primary object of the invention is to provide fine silicon-containing glass beads having a cavity therein and containing nanoparticles in a portion other than the cavity (hereinafter also referred to as “glass phase”), and a production method thereof. Another object of the invention is to provide silicon-containing glass beads comprising nanoparticles, which are identical to or different from those in the glass phase, and functional materials, such as pharmaceutical molecules, in the cavity (which does not contain silicon); and a production method thereof.

### Solution to Problem

A conventional method of producing nanoparticle-dispersed silicon-containing glass beads by a reverse micelle method (PTL 5) is illustrated in FIG. 1, for example. According to the reverse micelle method, a medium containing a hydrophobic organic solvent and a surfactant is mixed with a nanoparticle-dispersed aqueous solution containing partially hydrolyzed silicon alkoxide, thereby forming a reverse micellar solution (W/O-type emulsion). The nanoparticle-dispersed aqueous solution is contained in the aqueous phase of the reverse micelles. When silicon alkoxide is added to the reverse micellar solution, the alkoxide is first dispersed in the oil phase. The alkoxide is then brought into contact with the aqueous phase, thereby gradually being hydrolyzed to a higher hydrophilicity, and moves to the aqueous phase. Sol-gel reactions proceed there and the alkoxide is solidified into glass beads. At this time, the nanoparticles contained in the aqueous phase of the reverse micelle are simultaneously incorporated in the glass and dispersed in the glass bead.

However, this method can hardly introduce, in addition to nanoparticles, other nanoparticles, magnetic particles, and functional materials such as pharmaceutical molecules into fine glass beads.

The present inventors have conducted extensive research to solve the above problems. As a result, it was found that silicon-containing glass beads having a cavity therein, and containing nanoparticles A in the glass phase and a functional material in the cavity are obtained as follows: in the process of using a reverse micelle method to form silicon-containing glass beads in which semiconductor nanoparticles were dispersed, an aqueous solution containing partially hydrolyzed silicon alkoxide and nanoparticles A was added to a medium containing a hydrophobic organic solvent (e.g., cyclohexane) and a surfactant, and stirred to form a reverse micellar solution (W/O type emulsion); and then an alkaline aqueous solution of predetermined pH containing a functional material was added thereto and stirred. Additionally, it was confirmed that nanoparticles A and functional materials were introduced into the glass beads without the loss of their properties.

The method of the present invention is specifically illustrated in FIG. 2. Aqueous solution X containing partially hydrolyzed silicon alkoxide and nanoparticles A is added to a solution in which a surfactant is dispersed in the oil phase to form a solution (W/O type emulsion) of fine reverse micelles containing aqueous solution X. When alkaline aqueous solution Y containing a functional material is added to the solution, aqueous solution Y is first dispersed in the oil phase. After a period of time, a part of aqueous solution Y forms water droplets, which then move to the aqueous phase (aqueous solution X) of the fine reverse micelle.

Here, supposing that the hydrogen ion exponent of aqueous solution X containing nanoparticles A is represented as pH 1, and the hydrogen ion exponent of aqueous solution Y containing a functional material is represented as pH 2, silicon-containing glass beads having a cavity therein can be formed when the relation of pH 1 and pH 2 satisfies 7<pH 1<pH 2<14.

More specifically, the droplets of highly-alkaline aqueous solution Y that have entered into the aqueous phase (aqueous solution X) of the fine reverse micelle cause rapid progress of sol-gel reactions on the surface of aqueous solution X that is in contact with the droplets of aqueous solution Y to form glass shells while the form of the droplets is maintained in the reverse micelles; and they are then dried. The silicon-containing glass beads having a cavity therein are supposedly formed in this manner. If necessary, silicon alkoxide may be further added to the above reverse micelle solution, thereby promoting the sol-gel reactions.

Thus, nanoparticles A contained in aqueous solution X are dispersed and fixed in the glass phase, and the functional material contained in the droplets of aqueous solution Y is introduced into the cavity portion. As a result, nanoparticles A and the functional material are separately dispersed and fixed in different areas of one glass bead.

This method has the advantage that one kind of nanoparticles A are dispersed in the glass phase of a silicon-containing glass bead, and a functional material other than nanoparticles A is dispersed in the cavity (which does not contain silicon) of the glass bead. The silicon-containing glass beads of the present invention include those in which a functional material is dispersed along the inner surface of the cavity and the cavity remains in the center portion of the glass bead, and those in which the cavity is filled with a functional material. According to this method, nanoparticles A and the functional material do not come into direct contact with each other, and hence glass beads retaining the properties (fluorescent properties, magnetic properties, drug effects, etc.) inherent in nanoparticles A and the functional material prior to dispersion can be prepared.

More specifically, since the silicon-containing glass beads of the present invention have a cavity therein, and can contain nanoparticles A in the glass phase and a functional material in the cavity portion, fine glass beads that have both the functions of nanoparticles A and the functional material contained therein can be produced. In other words, glass beads each having several functions can be provided.

The present invention provides the following silicon-containing glass beads and a method of producing the same.

Item 1. Silicon-containing glass beads having an average particle diameter of 20 nm to 1 μm, each having one or more cavities therein, the silicon-containing glass beads containing nanoparticles A in a glass phase of each of said silicon-containing glass beads.
Item 2. The silicon-containing glass beads according to Item 1, further comprising a functional material in said one or more cavities.
Item 3. The silicon-containing glass beads according to Item 2, wherein the functional material is nanoparticles B.

Item 4. The silicon-containing glass beads according to Item 3, wherein said nanoparticles A and nanoparticles B each have an average particle diameter of 2 to 20 nm.

Item 5. The silicon-containing glass beads according to Item 3 or 4, wherein said nanoparticles A and/or nanoparticles B are semiconductor nanoparticles.
Item 6. The silicon-containing glass beads according to any one of Items 3 to 5, wherein said nanoparticles A and/or nanoparticles B are semiconductor nanoparticles with a PL efficiency of not less than 20%.
Item 7. The silicon-containing glass beads according to Item 5 or 6, wherein the semiconductor nanoparticles are at least one member selected from the group consisting of CdTe, CdSe, CdS, ZnSe, ZnSe₍₁₋ₓ₎Teₓ (0<x<1), ZnS, InP, InₓGa₍₁₋ₓ₎P (0<x<1), and InAs.
Item 8. The silicon-containing glass beads according to any one of Items 3 to 7, wherein said nanoparticles A and/or nanoparticles B are magnetic nanoparticles.
Item 9. The silicon-containing glass beads according to Item 8, wherein the magnetic nanoparticles are at least one member selected from the group consisting of Fe₃O₄, Fe₂O₃, CoFe₂O₄, MnFe₂O₄, NiFe₂O₄, CoCrFeO₄, Pt, Co, PtCo, FePt, and FeCo.
Item 10. The silicon-containing glass beads according to Item 8 or 9, which have a magnetization of 1 to 200 emu/g at an applied magnetic field of 5 kOe.
Item 11. The silicon-containing glass beads according to any one of Items 3 to 10, wherein said nanoparticles A and/or nanoparticles B are metal nanoparticles.
Item 12. The silicon-containing glass beads according to Item 11, wherein the metal of the metal nanoparticles is at least one member selected from the group consisting of gold (Au), silver (Ag), and copper (Cu).
Item 13. The silicon-containing glass beads according to any one of Items 1 to 12, wherein the silicon-containing glass beads have cavities having an average inner diameter of 10 to 500 nm.
Item 14. The silicon-containing glass beads according to any one of Items 2 to 13, wherein the functional material is a pharmacologically active substance.
Item 15. The silicon-containing glass beads according to any one of Items 1 to 14, wherein an antibody is attached to the outer surface of each silicon-containing glass bead.
Item 16. A method of producing silicon-containing glass beads having an average particle diameter of 20 nm to 1 μm, each having one or more cavities therein, the silicon-containing glass beads containing nanoparticles A in a glass phase of each of said silicon-containing glass beads, the method comprising the steps of:

(1) mixing a medium comprising a hydrophobic organic solvent and a surfactant with aqueous solution X comprising nanoparticles A and silicon alkoxide to prepare a reverse micellar solution; and

(2) adding alkaline aqueous solution Y to the reverse micellar solution prepared in step (1) to form the silicon-containing glass beads.

Item 17. The method according to Item 16, wherein in step (2), said alkaline aqueous solution Y contains a functional material.
Item 18. The method according to Item 16 or 17, wherein the hydrogen ion exponent of said silicon alkoxide aqueous solution X containing nanoparticles A (pH 1) of step (1) satisfies pH 1>7, and the hydrogen ion exponent of said alkaline aqueous solution Y (pH 2) of step (2) satisfies pH 1<pH 2<14.
Item 19. The method according to any one of Items 16 to 18, wherein step (2) comprising adding said alkaline aqueous solution Y to the reverse micellar solution prepared in step (1) and further adding thereto a silicon alkoxide to form the silicon-containing glass beads.
Item 20. A fluorescence reagent comprising the silicon-containing glass beads according to any one of Items 1 to 15.
Item 21. A drug delivery system comprising the silicon-containing glass beads according to any one of Items 1 to 15.

### ADVANTAGEOUS EFFECTS OF INVENTION

In the silicon-containing glass beads of the present invention having a cavity therein, nanoparticles A (e.g., semiconductor nanoparticles, magnetic nanoparticles, metal nanoparticles, etc.) may be dispersed in the glass phase, and nanoparticles B (e.g., semiconductor nanoparticles, magnetic nanoparticles, metal nanoparticles, etc.) and a functional material, such as a pharmacologically active substance, may be supported in the cavity portion. Since both nanoparticles A and the functional material are not mixed during the synthesis of the glass beads, the produced glass beads can retain fluorescent properties, magnetic properties, drug effects, and other properties of each substance. In other words, glass beads exhibiting the properties of each substance simultaneously can be produced.

Moreover, since glass is excellent in mechanical and chemical properties, these glass beads also exhibit excellent stability with the passage of time. For this reason, they can be used for purposes such as fluorescence reagents, antigen collection, and drug delivery (drug transportation in the human body).

### BRIEF DESCRIPTION OF DRAWINGS

FIG. 1 schematically illustrates a method of producing conventional nanoparticle-dispersed glass beads.

FIG. 2 schematically illustrates a method of producing the nanoparticle-dispersed glass beads having a cavity of the present invention. In this figure, nanoparticles B are dispersed in the cavity portion.

In FIG. 3, (a) shows an image of a CdTe nanoparticle-dispersed glass bead having a cavity (about 160 nm in particle diameter) photographed by the high angle annular dark field method (HAADF-STEM); and (b) shows an image of a CdTe nanoparticle-dispersed glass bead having cavities (about 900 nm in particle diameter) photographed under a transmission electron microscope.

FIG. 4 shows the absorption and fluorescence spectra of a colloidal solution of CdTe nanoparticles, and those of glass beads, in which CdTe nanoparticles were dispersed, in water.

FIG. 5 shows the absorption and fluorescence spectra of the glass beads with avidin attached in which the CdTe nanoparticles were dispersed.

FIG. 6 shows a transmission electron microscope photograph of a CdTe nanoparticle-dispersed glass bead in which magnetic nanoparticles (Fe₃O₄ nanoparticles) were dispersed in the cavity portion.

FIG. 7 shows an X-ray diffraction spectrum of the glass beads in which fluorescent nanoparticles (CdTe) and magnetic nanoparticles (Fe₃O₄) were dispersed.

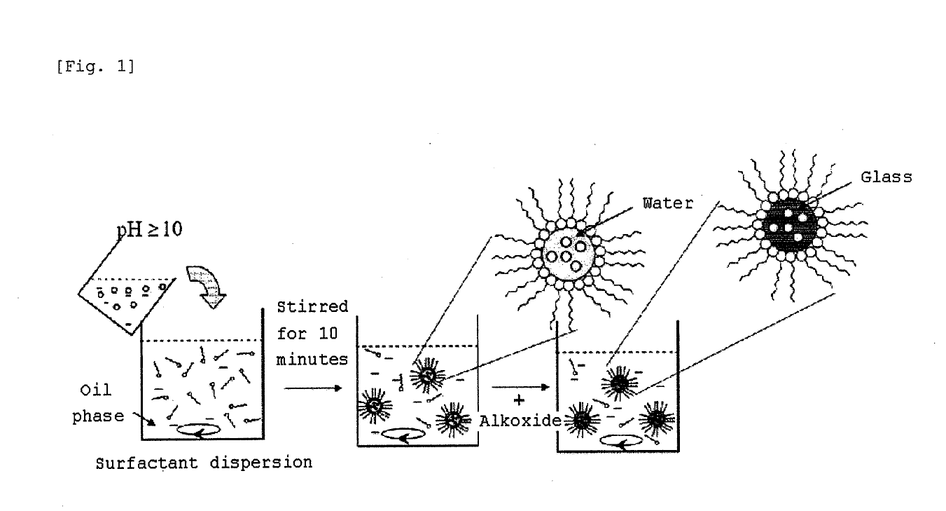

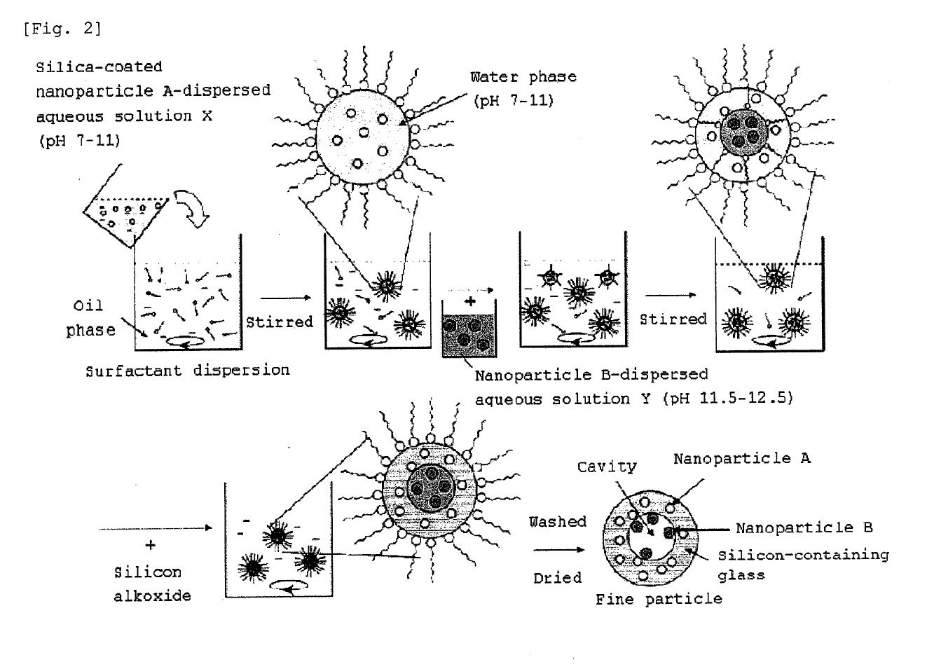

In FIG. 8, (a) shows the magnetic hysteresis curve of the glass beads in which fluorescent nanoparticles (CdTe) and magnetic nanoparticles (Fe₃O₄) were dispersed, and (b) shows the magnetic hysteresis curve of magnetic nanoparticles (Fe₃O₄) alone.

### DESCRIPTION OF EMBODIMENTS

The silicon-containing glass beads of the present invention having a cavity therein may contain nanoparticles A (semiconductor nanoparticles, magnetic nanoparticles, metal nanoparticles, etc.), which are dispersed in the glass phase, and a functional material, which is supported in the cavity portion. Examples of the functional material include nanoparticles B (semiconductor nanoparticles, magnetic nanoparticles, metal nanoparticles, etc.), pharmacologically active substances, etc. Nanoparticles A and nanoparticles B may be the same or different types.

Nanoparticles A and B (semiconductor nanoparticles, magnetic nanoparticles, metal nanoparticles, etc.), which can be introduced into the glass phase and/or the cavity portion, and their production methods are explained below. Subsequently, the silicon-containing glass beads containing nanoparticles A in the glass phase and a functional material (nanoparticles B, pharmacologically active substances, etc.) in the cavity portion, and their production method are explained.

### 1. Method of Producing Nanoparticles

The following explains nanoparticles used as nanoparticles A and B. Examples of such nanoparticles include semiconductor nanoparticles, magnetic nanoparticles, metal nanoparticles, and the like.

### 1.1 Semiconductor Nanoparticles

As semiconductor nanoparticles, those of Group II-VI and III-V are well known, and their production methods for use in the present invention are explained below. The production methods are devised for high PL intensity.

Preferred examples of nanoparticles A and B contained in the silicon-containing glass beads include semiconductor nanoparticles, magnetic nanoparticles, metal nanoparticles, etc., which are stable in an aqueous alkali solution. Preferred examples of semiconductor nanoparticles include those exhibiting PL.

### (1) II-VI Semiconductor Nanoparticles

Examples of II-VI semiconductor nanoparticles include those which are luminescent in the visible and infrared region, such as CdTe, CdSe, CdS, ZnSe, ZnSe₍₁₋ₓ₎Teₓ (0<x<1), ZnS, and other semiconductor nanoparticles. Other examples include PbS, PbSe, and the like. These can all be prepared by the known method explained below.

In one example of the production method using an aqueous solution, a II-VI semiconductor nanoparticles can be obtained by introducing a Group VI element compound into an alkaline aqueous solution, in which a surfactant and a water-soluble compound composed of a Group II element are dissolved, in an inert atmosphere. Group VI element compounds in gaseous form can also be used.

In this method, perchlorate is preferably used as the water-soluble compound composed of a Group II element. When the Group II element is cadmium, for example, cadmium perchlorate can be used.

As the surfactant, those having a thiol group (hydrophobic group) and a hydrophilic group are preferable. Examples of hydrophilic groups include anionic groups such as carboxyl groups; cationic groups such as amino groups; hydroxyl groups; etc. Particularly, anionic groups such as carboxyl groups are preferred. Specific examples of the surfactant include thioglycolic acid, thioglycerol, mercaptoethylamine, and the like.

As the Group VI element compound, for example, hydrides of Group VI elements, etc. can be used. When the Group VI element is tellurium, hydrogen telluride can be used. Alternatively, it is possible to introduce, as an aqueous solution, sodium hydrogen telluride obtained by reacting hydrogen telluride with sodium hydroxide.

During the synthesis process, high PL efficiency may be obtained when the amount of the surfactant used in the preparation of nanoparticles is about 1 to 1.5 in a molar ratio with respect to the Group II element contained in the aqueous solution.

As the inert atmosphere, atmospheres of gases that are uninvolved in the reaction are available. For example, atmospheres of argon gas, nitrogen gas, and other inert gases may be suitably used.

The above reaction can usually be performed at room temperature (e.g., about 10 to 30° C.). The pH of the alkaline aqueous solution is preferably about 10 to 12, and particularly preferably 10.5 to 11.5. The reaction is usually completed within about 10 minutes after the introduction of the Group VI compound.

The particle diameter of the semiconductor nanoparticles obtained in the above manner is of the nano meter order, and is mostly 2 to 20 nm. After the semiconductor nanoparticles are produced in the above manner, they are refluxed, and thereby the particle diameter of the semiconductor nanoparticles can be controlled. The longer the reflux time is, the larger the particle diameter becomes.

Particularly, a method of producing CdTe nanoparticles is explained in a document (Murase et al., Chemistry Letters, vol. 34, p. 92, 2005). Moreover, a method of producing ZnSe-containing nanoparticles is explained in a document (Murase et al., Journal of Colloid and Interface Science, vol. 321, p. 468 (2008)).

On the other hand, as the production method using an organic solution, for example, a method of adding a Group II element compound to a solution containing trioctylphosphine and oxides thereof, and further adding a Group VI element, is known. CdSe is the best-known nanoparticle produced by this method. Depending on the difference in the type of solvent and Group II element compound used, some reported cases are known, as described below.
Talapin et al., Nano Letters, vol. 1, p. 207 (2001)
Peng et al., Journal of the American Chemical Society, vol. 123, p. 1389 (2001)
Bawendi et al., Journal of Physical Chemistry B, vol. 101, p. 9463 (1997)
Peng et al., Journal of the American Chemical Society, vol. 124, p. 2049 (2002)
Zhong et al., Journal of Physical Chemistry C, vol. 111, p. 526 (2007)

### (2) III-V Semiconductor Nanoparticles

Suitable examples of III-V semiconductor nanoparticles include those having high PL efficiency in an alkaline aqueous solution, such as InP, InₓGa₍₁₋ₓ₎ P (0<x<1), InAs, and like semiconductor nanoparticles. Preferred examples include nanoparticles having a core/shell structure in which the core is composed of III-V nanoparticles, and the surface of the core is coated with a layer containing II-VI semiconductor. One example of the method of producing such nanoparticles is described below.

### (a) Production of a Core

As exemplified in a known document (Murase et al., Chemistry Letters, vol. 37, p. 856 (2008)), III-V semiconductor nanoparticles are synthesized at a relatively low temperature by using, as source of a Group V element, a compound having a partial structure in which two kinds of Group V atoms are directly bound to each other, and also using a solvothermal method (a procedure for synthesizing a desired product by placing a solvent in a pressure-resistant container, and adjusting the temperature not lower than the boiling point of the solvent). The temperature in the solvothermal method is about 100 to 300° C., more preferably about 120 to 200° C., and still more preferably about 150 to 180° C.

As the compound having a partial structure in which two kinds of Group V atoms are directly bound to each other, a compound having a partial structure in which a Group V atom and a nitrogen atom are directly bound to each other is suitably used. More specifically, the compound is represented by Formula (1):

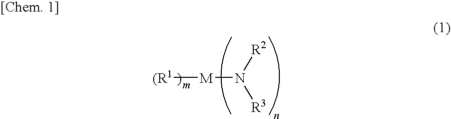

wherein M is a Group V element; R¹, R², and R³ are the same or different, and are each a lower alkyl group, such as methyl, ethyl, n-propyl, isopropyl, butyl, tert-butyl, and the like; m is an integer of 0 to 2; n is an integer of 1 to 3; and m+n=3.

Examples of the Group V element represented by M include P, As, Sb, Bi, etc.; and P is preferred. As the lower alkyl groups represented by R¹, R², and R³, C₁-C₃ alkyl is preferred, and methyl is more preferred. n is preferably 3.

Specific examples of the compound represented by Formula (1) include compounds represented by Formulae (2) to (7):

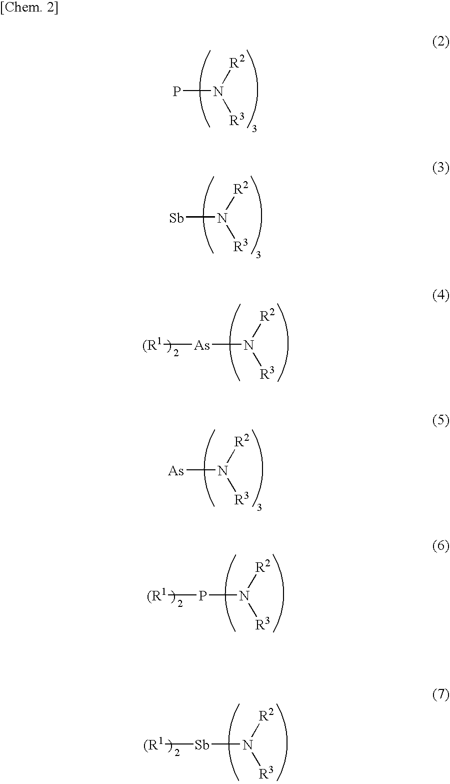

wherein M, R¹, R², and R³ are the same as defined above.

Among these compounds, those represented by Formulae (2), (3), and (5) are preferred. R¹, R², and R³ are each preferably methyl.

The source of a Group III element is not limited, and examples thereof include In, Ga, Al, and other Group III metals, and their chloride, oxide, nitrate, acetate, etc. Such Group III elements may be used singly or in a combination of two or more. Specific examples of Group III element compounds include InCl₃, In₂O₃, In(OOCCH₃)₃, AlCl₃, Al(NO₃)₃.9H₂O, GaCl₃, Ga(NO₃)₃.nH₂O; and InCl₃, AlCl₃, and GaCl₃ are preferred.

Examples of the solvent include toluene, chloroform, and other hydrophobic organic solvents.

According to this method, a poor solvent such as alcohol is added to the synthesized nanoparticles, thereby easily producing nanoparticles having a narrow size distribution (a size-selective precipitation method). The size-selective precipitation method is the procedure for classifying nanoparticles by gradually adding a poor solvent such as alcohol to the nanoparticles dispersed in a good solvent, so that the nanoparticles are gradually precipitated in order of solubility (generally starting from large-sized nanoparticles). The particle diameter of these nanoparticles is mostly about 1.5 to 6 nm. In order to increase the PL efficiency of the obtained III-V semiconductor nanoparticles, the surface of each particle (core) is further coated with a shell layer in the following manner.

### (b) Production of a Core/Shell Structure

(b-i) Preparation of a Mixed Solution

An organic solvent dispersion containing the III-V semiconductor nanoparticles (core) obtained above is brought into contact with an aqueous solution containing a compound composed of a Group II element and a compound composed of a Group VI element. At this time, a surfactant is dispersed in the aqueous phase. As a surfactant, water-dispersible molecules containing a Group VI element are favorably used. For example, thioglycolic acid is available. In this case, the sulfur atom at one end of the molecule is attached to the surface of each nanoparticle, and the carboxyl group at the other end of the molecule is ionized in the neutral to alkaline region, contributing to the stable dispersion of the nanoparticles in water. At this time, in addition to the surfactant, compounds containing other Group VI elements may be added.

This process results in elimination reactions of some Group III and V ions present on the surface of the III-V semiconductor nanoparticles. Thus, the Group III and V ions are substituted by Group II and VI ions in the aqueous solution, while the surfactant contained in the aqueous solution is bound to the surface of the nanoparticles. As a result, the nanoparticles contained in the organic solvent dispersion become hydrophilic and move to the aqueous phase, leading to a state where the III-V semiconductor nanoparticles are dispersed in the aqueous phase. Examples of the surfactant used here include thiols, amines, amino acids, etc. When thiols are used, since they contain sulfur, which is a Group VI element, the amount of other Group VI elements can be reduced. Examples of thiols include thioglycolic acid, thioglycerol, 2-mercaptoethylamine, 3-mercaptopropionic acid, mercaptosuccinic acid, and the like.

In the organic solvent dispersion containing III-V semiconductor nanoparticles, hydrophobic organic solvents may generally be used. Especially, aromatic hydrocarbon and halogenated hydrocarbon are suitably used. Specific examples thereof include toluene and chloroform.

The concentration of the III-V semiconductor nanoparticles in the organic solvent dispersion is, but not limited thereto, for example, preferably about 0.5×10⁻⁶ to 10×10⁻⁶ mol/L, more preferably about 1×10⁻⁶ to 6×10⁻⁶ mol/L, and even more preferably about 2×10⁻⁶ to 3×10⁻⁶ mol/L.

As the organic solvent dispersion, those in which the III-V semiconductor nanoparticles, which are obtained by the conventional hot soap method or the above-described solvothermal method, are dispersed may be used as they are. Alternatively, dispersions prepared by separating the III-V semiconductor nanoparticles, which are obtained using the hot soap method or the solvothermal method, from a solvent, and then dispersing the nanoparticles in the organic solvent again, may be used.

As the compound composed of a Group II element to be mixed in the aqueous solution, water-soluble compounds containing at least one kind of Group II element such as Zn, Cd, Hg, etc., can be used. Examples of such water-soluble compounds include perchlorate, chloride, nitrate, and the like.

The concentration of the Group II element ions in the aqueous solution is preferably about 0.01 to 0.3 mol/L, more preferably about 0.05 to 0.2 mol/L, and even more preferably about 0.065 to 0.15 mol/L.

As the compound composed of a Group VI element, water-soluble compounds containing at least one kind of Group VI element such as S, Se, Te, etc., can be used. Examples of such water-soluble compounds include thioglycolic acid, 3-mercaptopropionic acid, sodium sulfide, sodium sulfhydrate (NaSH), and the like. Additionally, as for selenide and telluride, similar compounds can be used.

The concentration of the Group VI element in the aqueous solution is preferably about 0.01 to 0.8 mol/L, more preferably about 0.05 to 0.5 mol/L, and even more preferably about 0.15 to 0.4 mol/L.

The molar ratio of the Group VI element to the Group II element ([Group VI element]/[Group II element]) in the aqueous solution is preferably about 1.5 to 4.0, more preferably about 1.8 to 3.0, and still more preferably about 1.9 to 2.5.

Moreover, when a thiol is introduced as a surfactant, it is added so that the total amount of the thiol and other Group VI element is within the above ratio. Examples of thiols include thioglycolic acid, thioglycerol, 2-mercaptoethylamine, 3-mercaptopropionic acid, mercaptosuccinic acid, and the like. The activity of the surfactant enhances the dispersibility of the nanoparticles in water.

The pH of the aqueous solution containing a compound composed of a Group II element and a compound composed of a Group VI element is preferably about 5 to 12, more preferably about 6.5 to 11.5, even more preferably about 9 to 10.

Any method can be used to bring the organic solvent dispersion containing the III-V semiconductor nanoparticles into contact with the aqueous solution containing a compound composed of a Group II element and a compound composed of a Group VI element. Either of the organic solvent dispersion and aqueous solution may be placed in a container, and the other may be added to the container. The reaction proceeds on the interface between the organic solvent dispersion and the aqueous solution. The progress of the reaction can be promoted by sufficiently mixing the organic solvent dispersion and the aqueous solution.

At this time, the volume ratio of the organic solvent dispersion containing the III-V semiconductor nanoparticles to the aqueous solution containing a compound composed of a Group II element and a compound composed of a Group VI element is preferably about 0.2 to 5, and more preferably about 0.7 to 1.3.

The solution temperature during mixing is preferably about 15 to 80° C., more preferably about 30 to 65° C., and most preferably about 45 to 55° C.

(b-ii) Light Irradiation

After the III-V semiconductor nanoparticles are transferred to the aqueous phase in the above manner and dispersed in the aqueous phase, the aqueous phase is irradiated with light. As a result of this process, a reaction of the Group II element with the Group VI element in the aqueous solution occurs on the surface of the III-V semiconductor nanoparticles to form II-VI semiconductor layers. In fact, this process is a competitive reaction of the dissolution of the nanoparticles by light irradiation and a supply of Group II and VI ions from the solution. Due to these reactions, the surface of each III-V nanoparticle (core) dissolves, while the surface is coated with a new II-VI semiconductor layer, thereby yielding nanoparticles having a core/shell structure. According to this process, the Group III element content in the nanoparticles is greater than the Group V element content. Moreover, this process greatly improves the PL efficiency, and thus water-dispersible nanoparticles having a high PL efficiency can be obtained.

When thioglycolic acid is used as the compound composed of a Group VI element in this case, the thioglycolic acid serves as the source of sulfur, which is a Group VI element, while the thiol group is bound to the surface of the formed shell portion, and the presence of the carboxyl group adds water dispersibility to the nanoparticles. When other thiols (thioglycolic acid, thioglycerol, 2-mercaptoethylamine, 3-mercaptopropionic acid, mercaptosuccinic acid, etc.) are used, the same mechanism stabilizes the nanoparticles.

In order to produce the effect of increasing the PL efficiency by light irradiation, the wavelength of light to be irradiated may be in the wave range where the nanoparticles have optical absorption properties. However, a wavelength that is too short is not desirable because it results in the effect of absorption of the solvent or surfactant. Moreover, small optical absorption is not preferred because the reaction proceeds slowly. Generally, light at a wavelength of about 300 to 600 nm is preferred, light at about 320 to 500 nm is more preferred, and light at about 330 to 400 nm is still more preferred.

The intensity of irradiation light is preferably about 0.1 to 6 W/cm², more preferably about 1 to 5 W/cm², and even more preferably about 3 to 4.5 W/cm².

The light irradiation time is generally about 1 minute to 12 hours, preferably about 20 minutes to 5 hours, and more preferably about 30 minutes to 2 hours, although the time is dependant on the irradiation intensity. Light irradiation under the above conditions greatly increases the PL efficiency of the nanoparticles, sometimes beyond 70%.

The thus obtained nanoparticles having a core/shell structure in which the core portion is composed of III-V semiconductor nanoparticles, and the shell portion is composed of a II-VI semiconductor can be precipitated and purified by adding a poor solvent such as alcohol. It is also possible to disperse them in water again.

The classification of the nanoparticles by adding a poor solvent and using the size-selective precipitation method may be conducted before water conversion, immediately before light irradiation, or immediately after light irradiation. Classification conducted after transfer of nanoparticles to water phase (immediately before or immediately after light irradiation) demonstrates a tendency to suppress surface oxidation and ensure a higher PL efficiency.

These semiconductor nanoparticles have a high PL efficiency. The nanoparticles sufficiently achieve PL efficiency higher than 20%, which is required for various applications. According to the III-V semiconductor nanoparticles, those having as high a PL efficiency as 50% or more in an aqueous solution can be obtained, although such a high PL efficiency was not achieved in the past. Particularly, those having a PL efficiency of 65% or more, and further those having as high as 70% or more can be obtained.

The “PL efficiency of the semiconductor nanoparticles in the solution” in this specification denotes the value normally used in the art to which the present invention pertains, and is synonymous with the “internal quantum yield”. This will be described below in detail in the following section of the method of producing glass beads in which nanoparticles having a cavity therein are dispersed.

### 1.2 Magnetic Nanoparticles

Nanoparticles having magnetic properties can also be produced by a known method. As a typical example, Fe₃O₄ nanoparticles can be synthesized in accordance with a previously published document (Stroeve et al., Chemistry of Materials, vol. 8, p. 2209 (1996)) using the following chemical formula.

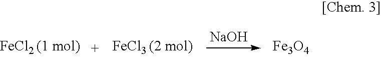

In this case, magnetic nanoparticles can be produced without the use of a surfactant in an alkaline solution (pH 11 to 12).

An overly high concentration of the magnetic nanoparticles in the alkaline solution results in aggregation. A concentration that is too low results in a reduced amount of nanoparticles that are finally incorporated into glass beads. Therefore, in the reaction of incorporating the nanoparticles into glass beads, the concentration of the nanoparticles is preferably 1×10⁻⁵ to 5×10⁻⁴ mol/L, and more preferably 5×10⁻⁵ to 2×10⁻⁴ mol/L.

In addition, at least one kind of magnetic nanoparticle selected from the group consisting of Fe₃O₄, Fe₂O₃ CoFe₂O₄, MnFe₂O₄, NiFe₂O₄, CoCrFeO₄, Pt, Co, PtCo, FePt, and FeCo can be synthesized in accordance with previously published documents. Some synthetic methods of these magnetic nanoparticles are explained in Williams et al., Accounts of Chemical Research, vol. 41, p. 411 (2008). As the magnetization (magnetic moment per unit volume, unit: emu/g) generated in a magnetic material upon the application of magnetic field is larger, the magnetic material is more strongly attracted to a magnet. The magnetic field of a commercially available permanent magnet is about 5 kOe (0.5 T in terms of magnetic flux density) at most. In order for the glass beads to be attracted to such a magnet, the glass beads must have a magnetization of at least 1 emu/g. On the other hand, Fe₃O₄ magnetic nanoparticles, which are often used as magnetic nanoparticles, alone have a magnetization of about 40 emu/g at an applied magnetic field of 5 KOe. Further, PtCo etc. having a laminated structure are expected to have about 10-fold magnetization. Accordingly, if PtCo is introduced into the glass beads, 200 emu/g of magnetization can be realized even at a weight fraction of 50%. Since the magnetic field 5 kOe can be easily generated using a commercially available electromagnet, the magnetization in this magnetic field can be easily measured using a small vibrating sample magnetometer. Although types of magnetism include paramagnetism, superparamagnetism, ferromagnetism, and the like, the nanoparticles may exhibit any type of magnetism.

### 1.3 Metal Nanoparticles

Metal nanoparticles can also be produced by a known method. Examples of the metal include gold (Au), silver (Ag), copper (Cu), etc. A typical example, gold nanoparticles, can be synthesized in accordance with a previously published document (Nie et al., Journal of American Chemical Society, vol. 124, p. 9606 (2002)) by reduction of chloroauric acid. The particle diameter can be controlled in the range of 2 to 9 nanometers. Moreover, silver nanoparticles can be synthesized in accordance with a previously published document (Pal et al., Journal of Physical Chemistry C, vol. 112, p. 4874 (2008)).

### 2. Production of Glass Beads in which Nanoparticles Having a Cavity Therein are Dispersed

The silicon-containing glass beads of the present invention have an average particle diameter of 20 nm to 1 μm, have a cavity therein, and contain nanoparticles A in the glass phase. A typical example of their production method comprises the steps of:

(1) mixing a medium containing a hydrophobic organic solvent and a surfactant with aqueous solution X containing nanoparticles A and silicon alkoxide to prepare a reverse micellar solution; and

(2) adding alkaline aqueous solution Y to the reverse micellar solution of step (1) to form silicon-containing glass beads.

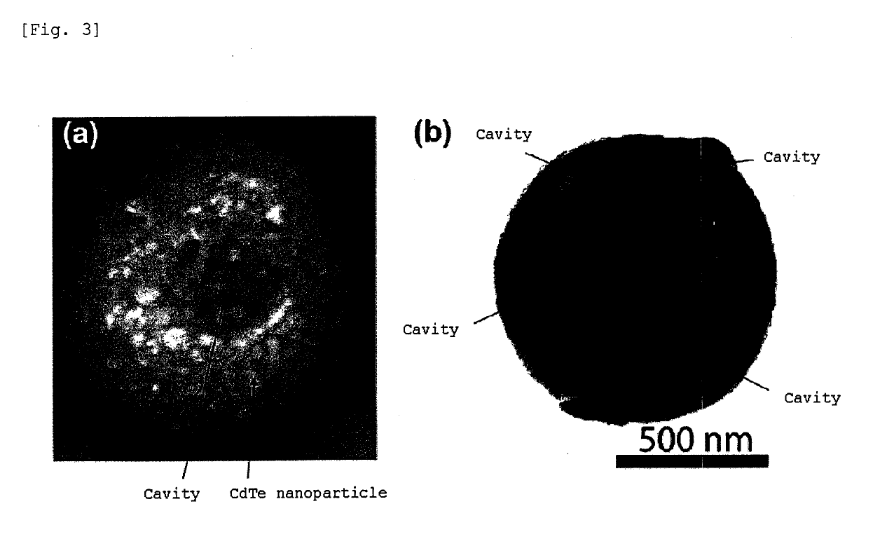

Each step is specifically explained below with reference to FIG. 2.

### Step (1)

In step (1), a reverse micellar solution is prepared by mixing a medium containing a hydrophobic organic solvent and a surfactant with aqueous solution X containing nanoparticles A and silicon alkoxide (FIG. 2).

Examples of the hydrophobic organic solvent used in step (1) include C₄-C₁₂ hydrocarbons, and specific examples include C₄-C₁₂ linear, branched, or cyclic aliphatic hydrocarbons and C₆-C₁₂ aromatic hydrocarbons. Such aliphatic hydrocarbons may be saturated or unsaturated as long as their melting and boiling points are not in the range of 10 to 35° C., and they are in liquid form at room temperature. Preferred are C₅-C₁₀ linear, branched or cyclic saturated aliphatic hydrocarbons. More specific examples include pentane, cyclopentane, hexane, cyclohexane, heptane, isoheptane, octane, isooctane, nonane, decane, and the like. Particularly, cyclohexane and isooctane are preferred. These aromatic hydrocarbons are monocyclic or bicyclic aromatic hydrocarbons, which may have an aliphatic hydrocarbon group on the aromatic ring. More specific examples include benzene, toluene, xylene, etc.

As the surfactant used in step (1), those which can generate the so-called reverse micelles can be used. Reverse micelles are such that in the state where a surfactant is dissolved in a hydrophobic organic solvent, the hydrophobic group side of the surfactant is oriented outward, and the hydrophilic group side of the surfactant is oriented inward.

Examples thereof include ionic (cationic, anionic, or amphoteric) surfactants, in which hydrophilic groups and hydrophobic groups are electrically charged, and nonionic surfactants, in which hydrophilic groups and hydrophobic groups are not electrically charged.

As ionic (anionic) surfactants, bis(2-ethylhexyl)sodium sulfosuccinate (trade name: “Aerosol OT”; manufactured by Wako Pure Chemical Industries, Ltd.) etc. are exemplified, for example.

As nonionic surfactants, polyoxyethylene ether type nonionic surfactants are exemplified, for example, and particularly, polyoxyethylene alkyl phenyl ether type nonionic surfactants are available. Among these, preferred examples include polyoxyethylene nonylphenyl ether type nonionic surfactants, such as polyoxyethylene(5)nonylphenyl ether (trade name: “Igepal CO-520”; manufactured by Sigma-Aldrich Corporation), polyoxyethylene(2)nonylphenyl ether (trade name: “Igepal CO-210”; manufactured by Sigma-Aldrich Corporation), polyoxyethylene(9)nonylphenyl ether (trade name: “Igepal CO-630”; manufactured by Sigma-Aldrich Corporation), polyoxyethylene(12)nonylphenyl ether (trade name: “Igepal CO-720”; manufactured by Sigma-Aldrich Corporation), etc.; polyoxyethylene isooctylphenyl ether type nonionic surfactants, such as polyoxyethylene(2)isooctylphenyl ether (trade name: “Igepal CA-210”; manufactured by Sigma-Aldrich Corporation), polyoxyethylene(5)isooctylphenyl ether (trade name: “Igepal CA-520”; manufactured by Sigma-Aldrich Corporation), polyoxyethylene(12)isooctylphenyl ether (trade name: “Igepal CA-720”; manufactured by Sigma-Aldrich Corporation), etc.; and the like.

The number in parenthesis described after each polyoxyethylene denotes the number of repetitions of the oxyethylene unit.

Nonionic surfactants are preferred, polyoxyethylene ether type nonionic surfactants are more preferred, polyoxyethylene nonylphenyl ether type nonionic surfactants are particularly preferred, and polyoxyethylene(5)nonylphenyl ether is even more preferred. The reason is considered to be that nanoparticles are easily incorporated into reverse micelles uniformly because electrostatic repulsion does not occur between the reverse micelles and nanoparticles, as stated above.

A medium containing a hydrophobic organic solvent and a surfactant may be prepared by mixing and stirring both. The amount of the surfactant used may be about 0.001 to 0.1 mol, and preferably about 0.005 to 0.02 mol, based on 1 mol of hydrophobic organic solvent. The temperature during stirring is usually, but is not limited to, about 10 to 35° C.

Aqueous solution X containing nanoparticles A and silicon alkoxide is prepared by mixing a nanoparticle A-dispersed aqueous solution with silicon alkoxide. The silicon alkoxide is represented by Formula (I):

R⁴ᵣ—Si(OR⁵)₄₋ᵣ (I)

wherein R⁴ and R⁵ are the same or different, and are each a lower alkyl group; and r is 0, 1, 2, or 3.

Among the compounds represented by Formula (1), preferred are those in which r is 0, 1, or 2, and particularly preferred are those in which r is 0 or 1.

Examples of the lower alkyl groups represented by R⁴ and R⁵ include C₁-C₆ linear or branched alkyl groups. Specific examples include methyl, ethyl, n-propyl, isopropyl, etc.; and methyl and ethyl are particularly preferred.

Preferred examples of the compound represented by Formula (I) include tetramethoxysilane, tetraethoxysilane (TEOS), tetraisopropoxysilane, methyltrimethoxysilane, ethyltrimethoxysilane, etc.; and TEOS is particularly preferred.

The amount of silicon alkoxide may be such that the number of moles of silicon alkoxide to the number of moles of semiconductor nanoparticles is about 1000:1 to 100000:1, and preferably about 5000:1 to 20000:1. The number of moles of semiconductor nanoparticles denotes that the number of semiconductor nanoparticles (not the number of semiconductor molecules) divided by the Avogadro's number is used as the number of moles. The molar absorption coefficient of semiconductor nanoparticles is determined by the material and size of the nanoparticles, and is reported in many documents. For example, CdSe, CdTe, and CdS nanoparticles are described in detail in a document (Yu et al., Chemistry of Materials, vol. 15, p. 2854 (2003)). Further, CdTe nanoparticles of a specific size are described in supplemental data of a document (Murase et al., Nanoscale Research Letters, vol. 2, p. 230 (2007)). Utilizing methods of these documents, the molar concentration of semiconductor nanoparticles can easily be calculated from the absorbance of a target solution. Further, if the volume of aqueous solution X to be applied is clarified, the number of moles of the semiconductor nanoparticle contained therein can be calculated.

In the aqueous solution dissolving silicon alkoxide, the mixing ratio (molar ratio) of silicon alkoxide to water is generally about 1:100 to 1:10000, preferably about 1:200 to 1:2000, and more preferably about 1:300 to 1:1000.

In the aqueous solution containing nanoparticles A and silicon alkoxide obtained by the above mixing process, the silicon alkoxide is partially hydrolyzed. The partial hydrolysis of silicon alkoxide is achieved by stirring the aqueous solution containing nanoparticles A and silicon alkoxide.

At this time, an alkaline aqueous solution may be added, as necessary. As an alkaline aqueous solution, for example, aqueous solutions of alkali metal hydroxide, such as sodium hydroxide, potassium hydroxide, etc., may be suitably used, in addition to ammonia water. These alkaline aqueous solutions can accelerate the partial hydrolysis of alkoxide. Among these, ammonia water or an aqueous sodium hydroxide solution is particularly preferred.

The amount of the alkaline aqueous solution added is not limited. For example, it may be added to the nanoparticle A-dispersed aqueous solution so that the pH of the nanoparticle A-dispersed aqueous solution is about 7 to 11 (preferably about 8.5 to 10).

Moreover, in addition to these alkaline aqueous solutions, metal ions constituting nanoparticles A and/or a surfactant for coating nanoparticles A may be added for the purpose of suppressing deterioration of the nanoparticles.

Examples of the metal ions constituting nanoparticles A include metal ions of the above-described semiconductor nanoparticles, and specific examples include zinc ions and cadmium ions.

The surfactant for coating nanoparticles A is not limited as long as it has the property of coating the surface of nanoparticles A. Specific examples thereof include at least one member selected from the group consisting of thioglycolic acid, thioglycerol, 2-mercapto-ethylamine, glycine, and the like. Among these, thioglycolic acid is preferred.

As aqueous solution X, it is necessary to obtain a solution in which silicon alkoxide is hydrolyzed partially, not completely. In the solution containing partially hydrolyzed alkoxide and nanoparticles A, nanoparticles A are presumably stabilized due to a silica coating on the surface of the particles. That is, the coating of nanoparticles A with thin glass layers prevents aggregation of nanoparticles A. Moreover, by using partially hydrolyzed products of silicon alkoxide with increased viscosity, semiconductor nanoparticles can be incorporated into reverse micelles, and then hydrolysis of the silicon alkoxide is immediately advanced to achieve vitrification.

The stirring temperature is generally, but not limited to, around room temperature (5 to 50° C.), and preferably 10 to 40° C. The time for partial hydrolysis of silicon alkoxide (stirring time) in step (1) is not limited, but is generally 1 to 6 hours, and preferably 2 to 4 hours.

Subsequently, aqueous solution X containing nanoparticles A and silicon alkoxide is mixed and stirred in the medium containing a hydrophobic organic solvent and a surfactant, thereby preparing a reverse micellar solution (FIG. 2).

The stirring temperature is generally, but not limited to, around room temperature (5 to 50° C.), and preferably 10 to 40° C. The stirring time is not limited, but generally 1 to 6 hours, and preferably 2 to 4 hours.

In order to generate reverse micelles of uniform size, the solution must be stirred vigorously, and thereby fine reverse micelles having a mean diameter (outside diameter) of about 50 to 5000 nm are formed. The mean diameter (outside diameter) of the reverse micelles is variable depending on the relative proportion between the amounts of the surfactant, water, and hydrophobic organic solvent.

### Step (2)

In step (2), silicon-containing glass beads are formed by adding alkaline aqueous solution Y to the reverse micellar solution obtained by step (1). Alkaline aqueous solution Y may contain the above-described functional materials (nanoparticles B, pharmacologically active substances, etc.).

Here, when the hydrogen ion exponent of aqueous solution X containing nanoparticles A is represented as pH 1, and the hydrogen ion exponent of alkaline aqueous solution Y optionally containing a functional material is represented as pH 2, it is critical that the relation between pH 1 and pH 2 satisfy 7<pH 1<pH 2<14. This enables the formation of silicon-containing glass beads having a cavity therein.

The difference between pH 2 and pH 1 is preferably 0.5 or greater, more preferably 1.0 or greater, and most preferably 1.5 to 2.5. This is because the rates of hydrolysis and dehydration condensation of alkoxide are appropriate in this range.

The range of pH 1 is preferably 7 to 10.5, more preferably 8 to 10.3, and most preferably 9 to 10.2. The range of pH 2 is, in addition to the above conditions, preferably 7.5 to 14, more preferably 10 to 13, and most preferably 11.5 to 12.5. These ranges are preferable because the higher alkalinity of pH 2 may cause glass dissolution.

Nanoparticles B are not limited as long as they are stable in the alkaline region (pH>7). For example, the above-described magnetic nanoparticles, metal nanoparticles, etc., can be used. Moreover, an aqueous solution in which organic molecules or inorganic materials stable in the alkaline region are dispersed can also be used. When such organic molecules and inorganic materials are pharmacologically active substances (drugs), they can be dispersed in glass beads without loss of their drug effects.

It is preferable that the amount of aqueous solution X containing nanoparticles A and silicon alkoxide (volume: Wₓ), which is added in step (1), be greater than the amount of alkaline aqueous solution Y optionally containing a functional material (volume: W_{y}). More specifically, the volume ratio of both solutions (Wₓ/W_{y}) is preferably about 3 to 100, more preferably about 10 to 40, and most preferably about 15 to 25. In this range, the aqueous phase (droplets) of aqueous solution Y, which is smaller than the aqueous phase (droplets) of aqueous solution X, can be introduced into the aqueous phase (droplets) of aqueous solution X, which is formed in the inside of reverse micelles, thus forming silicon-containing glass beads having a desired cavity.

According to this method, as illustrated in FIG. 2, when alkaline aqueous solution Y is added to a solution of fine reverse micelles encapsulating aqueous solution X containing partially hydrolyzed silicon alkoxide and nanoparticles A (W/O type emulsion), aqueous solution Y is first dispersed in the hydrophobic organic solvent (oil phase). After a while, aqueous solution Y is partially formed into droplets, which then move into the aqueous phase (aqueous solution X) encapsulated in the fine reverse micelles. The alkaline solution may contain a functional material.

When the droplets of highly alkaline aqueous solution Y enter into the aqueous phase (aqueous solution X) in the fine reverse micelles, sol-gel reaction proceeds rapidly on the surface of aqueous solution X in contact with the droplets of aqueous solution Y, and glass shells are formed while maintaining the droplet form in the reverse micelles. After drying them, silicon-containing glass beads having a cavity therein are considered to be prepared.

If necessary, silicon alkoxide may be further added to the above reverse micellar solution.

By this method, one kind of nanoparticles A can be dispersed in the glass phase of a silicon-containing glass bead, and other kinds of functional materials can be dispersed in the cavity of the glass bead. Since nanoparticles A and functional materials do not come into direct contact with each other, this method enables the production of glass beads that maintain the properties (fluorescent properties, magnetic properties, drug effects, etc.) inherent in nanoparticles A and the functional materials before dispersion.

In step (2), the stirring temperature is generally, but not limited to, around room temperature (5 to 50° C.), and preferably 10 to 40° C. The time for hydrolysis of silicon alkoxide (stirring time) is not limited but is generally 1 to 6 hours, and preferably 2 to 4 hours. When the reaction is completed thereafter, silicon-containing glass beads are formed in the hydrophobic organic solvent.

The solvent is removed from the liquid medium that prepares the silicon-containing glass beads having a cavity therein (and optionally containing a functional material in the cavity), obtained in step (2), to obtain the target silicon-containing glass beads. If necessary, the excess surfactant attached to the surface of the generated silicon-containing glass beads may be washed with a suitable solvent, and the silicon-containing glass beads may be dried.

The form of the obtained silicon-containing glass beads is almost spherical (including a true sphere, oval sphere, etc., for example), and the mean particle diameter thereof is about 20 nm to 1 μm. Each silicon-containing glass bead may have one or more cavities, and generally has 1 to 10 cavities. The average inner diameter of cavities is preferably 10 to 500 nm. The mean particle diameter of glass beads and the average inner diameter of cavities can be measured by the high angle annular dark field (scanning transmission electron microscope) method (HAADF-STEM) and transmission electron microscope (TEM).

The particle diameter D of the glass beads produced by this method can be reduced to below 1 μm, and at minimum, down to about 20 nm. Due to this, the glass beads can be used in fluorescent reagents, drug transport (drug delivery) in the living body, etc. Moreover, the diameter d of the cavity of the beads is dependent on the ratio of alkaline solution Y to X and the stirring rate, and the beads can be produced in the range of D/10≦d≦D/2. It is also possible to form not only one cavity but also a plurality of cavities in each glass bead. For this purpose, when the volume of alkaline solution X is represented as Wₓ, and the volume of alkaline solution Y is represented as W_{y}, 10≦Wₓ/W_{y}≦40 may be satisfied. Under this condition, glass beads that have about 2 to 10 fine cavities can be produced. When the total volume of the cavities in one glass bead is represented as V_{d}, V_{d} and remaining volume V_{D} can be adjusted in the range of 8≦V_{D}/V_{d}≦1000. Thus, as described above, the nanoparticles previously contained in aqueous solution X can be dispersed in volume V_{D}. The glass beads and cavities therein may not take the form of a perfect sphere. When they take the form of an oval, the particle diameter of the particles is defined by the average of three particle diameters in the directions of principal axes of inertia.

As previously explained, examples of nanoparticles include those producing fluorescence, those exhibiting magnetic properties, those having an intense plasmon absorption as metal, and the like. Moreover, examples of organic substances include pharmacologically active substances (drugs), which can be arranged in a specific area in the body to deliver a drug intensively to a target area, and slowly release the drug utilizing the properties of glass for effective action.

Moreover, antibodies can be attached to the glass surface to bond specific antigens in a specimen. When fluorescent semiconductor nanoparticles and magnetic nanoparticles are dispersed in the glass beads, the beads to which the antigens are attached can be collected by a magnet, and their type can be determined on the basis of fluorescent colors. At this time, it is desirable that the PL efficiency of the semiconductor nanoparticles is not decreased even when they are incorporated into the glass beads. A PL efficiency of not less than 20% is practically preferred.

The PL efficiency of the glass beads in the present specification is the value normally used in the art to which the present invention pertains, as previously described in the explanation of fluorescent semiconductor nanoparticles, and is synonymous with the “internal quantum yield”. This is defined as the ratio (Φ_{PL}/Φ_{A}) of the number of photons emitted as photoluminescence (Φ_{PL}) to the number of absorbed photons (Φ_{A}). If there is no aggregation and ignorable scattering, the PL efficiency is measured using the state of the solution. In this case, the PL efficiency is calculated by using known dye molecules and comparing the absorbance and PL intensity at an excitation light wavelength in the dye molecule solution and a target for measurement. In the measurement, the absorbance at an excitation wavelength is generally conformed between a dye molecule solution (e.g., aqueous solution of quinine sulfate in 0.05 mol/L sulfuric acid) and a target for measurement before comparison. PL efficiencies of powders, plates, and thin films are ordinarily measured using the integrating sphere, and devices for this purpose are commercially available (e.g., C9920 available from Hamamatsu Photonics K.K.). Details of the measurement of PL efficiency in the present specification are described in the latest works of the inventors (Murase et al., Journal of Luminescence, vol. 128, p. 1896, (2008)). This method allows an accurate measurement of the PL efficiency without consistent absorbance at an excitation wavelength.

### EXAMPLES

The present invention is described in more detail below with reference to examples; however the present invention is not limited thereto. The PL efficiencies of the solutions were measured at an excitation of 400 nm using aqueous solution of quinine sulfate in 0.05 mol/L sulfuric acid (PL efficiency: 55%) as a standard solution.

### Example 1

### Production of Glass Beads Having a Cavity in Which Fluorescent Semiconductor Nanoparticles are Dispersed)

### (1) Production of CdTe Nanoparticles

CdTe nanoparticles, i.e., semiconductor nanoparticles, were synthesized in accordance with a previously published document (Murase et al., Chemistry Letters, vol. 34, p. 92 (2005)). More specifically, 1.095 g (2.61 mmol) of cadmium perchlorate hexahydrate (Cd(ClO₄)₂.6H₂O) was dissolved in 200 mL of pure water, and thioglycolic acid, i.e., a surfactant for nanoparticles, was further added at a molar ratio of thioglycolic acid to cadmium of 1.3. After adjusting the pH to 11.4, the mixture was deaerated with argon gas, and then hydrogen telluride gas was introduced. The amount of tellurium in the gas was adjusted so as to be 0.47 times the amount of cadmium. Subsequently, the mixture was refluxed at 100° C. for 100 hours, thereby producing red-emitting CdTe nanoparticles (about 4 nm in particle diameter). The PL efficiency of the nanoparticles was about 72%.

### (2) Coating of Nanoparticles with a Silica Layer

Taking out 2 mL of the above CdTe nanoparticle dispersion in a concentration of 2.2×10⁻⁶ mol/L, an aqueous ammonia solution (6.25 wt. %, 50 μL) and TEOS (20 μL) were added and stirred for 2 hours. As a result, an aqueous solution in which the nanoparticles coated with a thin silica layer were dispersed (X1, pH 10) was produced.

### (3) Production of a Reverse Micellar Solution

A surfactant (Igepal CO-520; 3.52 g) was added to 25 g of cyclohexane and stirred until the solution became transparent. The nanoparticle-dispersed aqueous solution (X1) produced above was taken in an amount of 2 mL and added dropwise to the solution while vigorously stirring. Further, an aqueous ammonia solution (Y1) (6.25 wt. %, pH: about 12.3, 100 μL) was added.

### (4) Production of Glass Beads Having a Cavity

TEOS (150 μL) was further added to the reverse micellar solution prepared above and stirred for 24 hours.

As a result, a dispersion of silicon-containing glass beads having a cavity and containing CdTe nanoparticles dispersed in the glass phase was produced. At this stage, the glass beads had a wide size distribution ranging from about 50 nm to 2 μm.

Next, the size of the glass beads was selected by the procedure of filtering and centrifugation.

In order to obtain small-sized beads, the dispersion was first filtered through a filter with a 0.45 μm-pore size, and the filtrate was centrifuged at 22000 rpm for 30 minutes. The precipitate was washed with ethanol and then dispersed in pure water.

When the glass bead obtained in this manner was photographed by the high angle annular dark field (scanning transmission electron microscope) method (HAADF-STEM), the resulting image was as shown in FIG. 3 (a). It was confirmed that many CdTe nanoparticles were dispersed in the glass phase portion of the glass sphere with a diameter of about 160 nm. The presence of a cavity portion was also confirmed.

In order to obtain large-sized beads, the dispersion was first filtered through a filter with a 5 μm-pore size. The dispersion after filtering was then filtered through a filter with a 1 μm-pore size. The solid remaining on the upper surface of the filter was washed with ethanol, then dispersed again in water, and filtered through a filter with 1 μm pore size. This operation was repeated 10 times. When the glass bead obtained in this manner was observed under a transmission electron microscope, the resulting image was as shown in FIG. 3 (b). As indicated by arrows, at least four cavities are observed in the periphery of the glass bead with a diameter of 900 nm. Since an electron beam is difficult to pass through such a large-sized glass bead, cavities near the center of the bead are not observable. Moreover, CdTe nanoparticles (about 4 nm in diameter) are not observable at such a low magnification.

### Example 2

### Production of Glass Beads in which Fluorescent Semiconductor Nanoparticles and Magnetic Particles are Dispersed

### (1) Production of CdTe Nanoparticles and Fe₃O₄ Nanoparticles

Red-emitting CdTe nanoparticles (4 nm in particle diameter) were obtained in the same manner as in Example 1. The prepared CdTe nanoparticles were redispersed in an aqueous solution of 0.005 mol/L of thioglycolic acid (a surfactant for semiconductor nanoparticles) and 0.0017 mol/L of cadmium perchlorate. The pH was adjusted to 10, and the concentration of the nanoparticles was adjusted to 2.2×10⁻⁶ mol/L. Taking this dispersion in an amount of 2 mL, an aqueous ammonia solution (50 μL, 6.25 wt. %) and TEOS (20 μL) were added and stirred for 2 hours in a covered container. As a result, a dispersion of nanoparticles with thin silica coating (X2, pH 10) was prepared.

In addition, Fe₃O₄ nanoparticles (8 nm in particle diameter) were synthesized in accordance with a previously published document (Stroeve et al., Chemistry of Materials, vol. 8, p. 2209 (1996)). More specifically, 0.10 mL of 12N hydrochloric acid and 5 mL of deaerated pure water were mixed, and 0.1038 g of iron (II) chloride and 0.26 g of iron (III) chloride were further added and dissolved while stirring. This solution was added to 25 mL of sodium hydroxide solution (1.5 mol/L) while vigorously stirring. While attracting the generated precipitate using a magnet, the supernatant was removed by decantation. Pure water was added to the separated precipitate, the mixture was centrifuged at 4000 rpm, and the supernatant was removed. After repeating this operation 3 times, 500 mL of hydrochloric acid (0.01 mol/L) was added while stirring, and the negative charge on the surface was removed. Then, the mixture was further centrifuged at 4000 rpm, and redispersed in pure water, thus producing a Fe₃O₄ nanoparticle dispersion.

### (2) Production of a Reverse Micellar Solution

Igepal CO-520 (a surfactant for glass beads; 3.52 g) was added to 25 g of a cyclohexane solution, and stirred until the mixture became transparent. The nanoparticle dispersion (X2, pH 10; 2 mL) prepared above was added drop by drop thereto while vigorously stirring. A solution (Y2, ammonia: 6.25 wt. %, pH 12.3, 100 μL), which was prepared by adding 0.1 mL of aqueous ammonia (25 wt. %) to 0.3 mL of the iron oxide solution synthesized above, was further added drop by drop thereto.

### (3) Production of Glass Beads

TEOS (150 μL) was added to the above reverse micellar solution and stirred for 24 hours, proceeding the reaction.

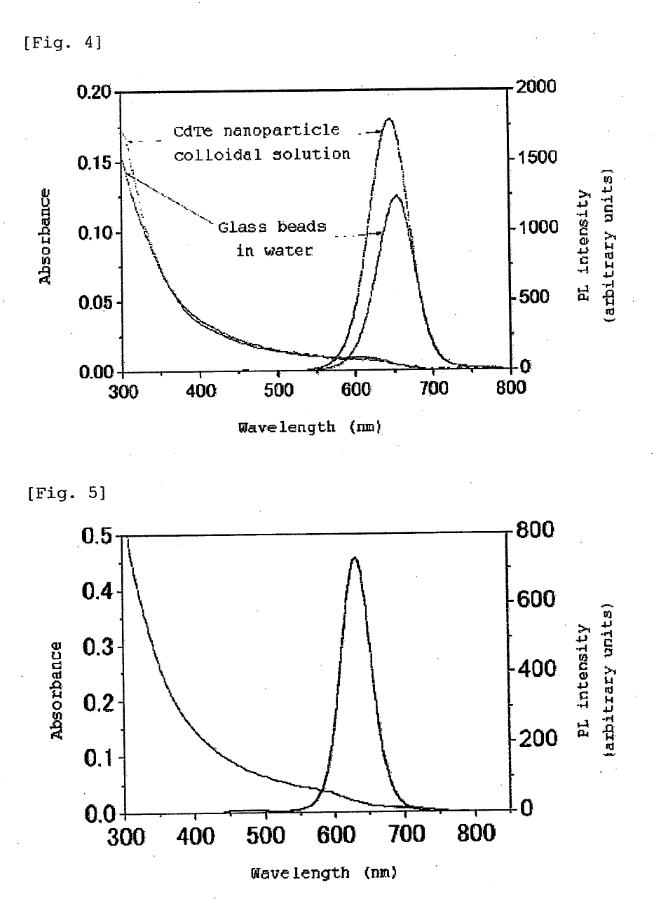

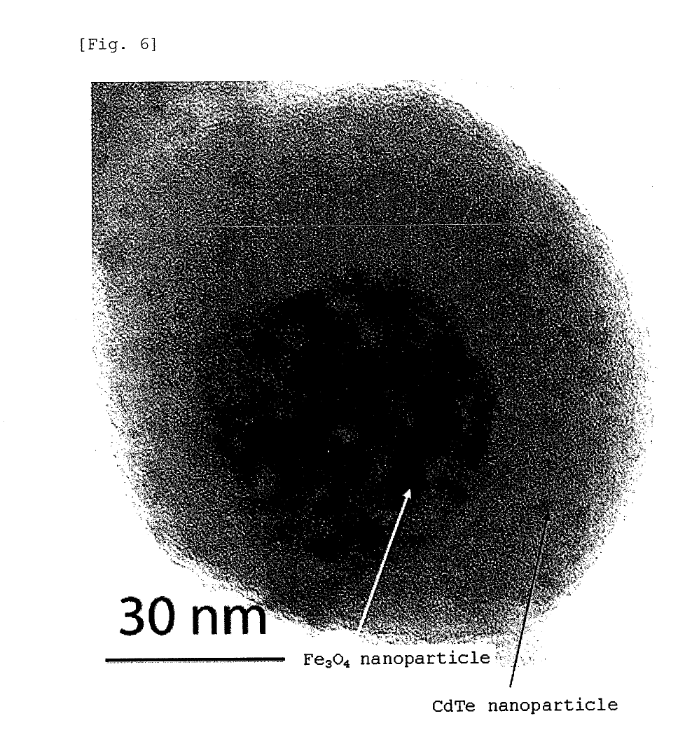

The thus obtained glass beads were attracted to a magnet. Further, they emitted intense red PL. As shown in FIG. 4, the absorption and PL spectra observed were almost the same as those of the initial CdTe nanoparticle colloidal solution. The PL efficiency was 68%. It was also found that when avidin-biotin was further attached through polyethylene glycol, similar absorption and PL spectra were indicated, as shown in FIG. 5.

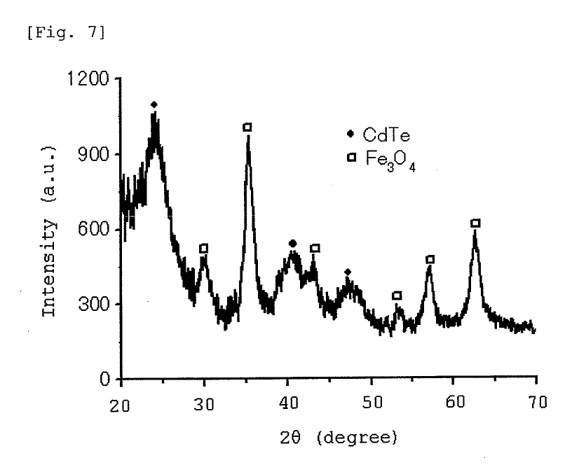

When the glass bead was observed under a transmission electron microscope, it was recognized that many of the CdTe nanoparticles were dispersed in the glass phase, as shown in FIG. 6. It was also confirmed that the Fe₃O₄ nanoparticles were dispersed in a cluster in the cavity portion.

After the glass beads were dispersed in the aqueous solution and put in a small glass bottle, a neodymium magnet with a surface magnetic flux density of 0.45 T was brought closer to the bottle. Then, it was observed that the glass beads were attracted to the magnet.

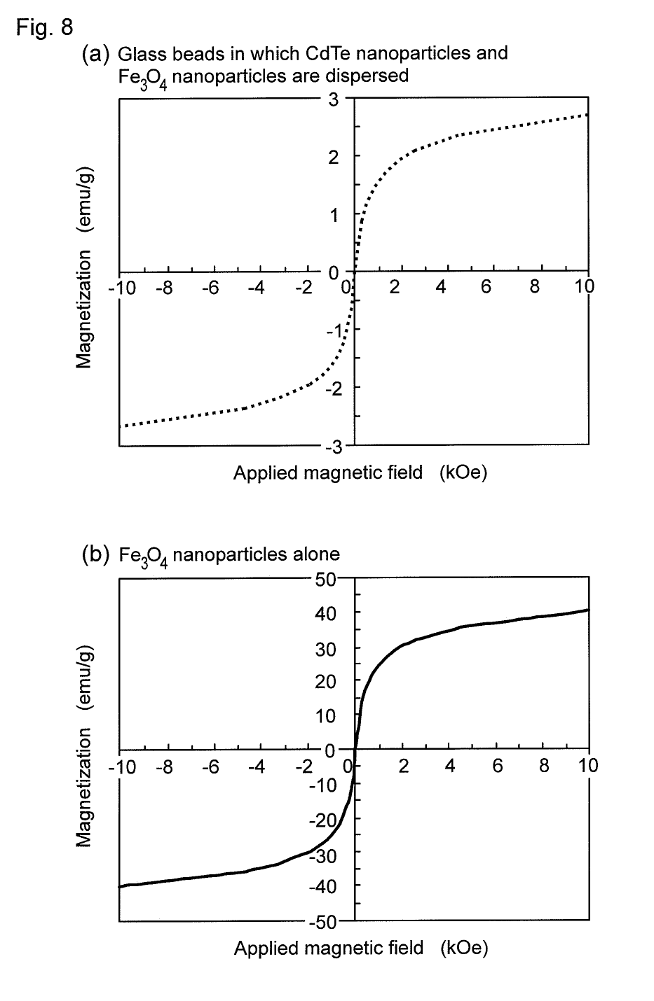

Subsequently, the glass beads were dried, and the X-ray diffraction spectra were measured. The results were as shown in FIG. 7. The angles of diffraction peaks indicated by filled circles were all consistent with the values of CdTe in the documents, and the angles of diffraction peaks indicated by outline squares were all consistent with the value of Fe₃O₄ in the documents. Accordingly, the dispersed nanoparticles were confirmed to be CdTe nanoparticles and Fe₃O₄ nanoparticles. Moreover, a wide diffraction peak appeared at about 22 degrees, which is typical in glass. The results also revealed that the matrix in which the nanoparticles were dispersed showed the properties of glass.

Moreover, the magnetic hysteresis curve (horizontal axis: applied magnetic field, vertical axis: magnetization) of the glass beads and that of the Fe₃O₄ nanoparticles alone were measured. The measurement was performed at room temperature using a vibrating sample magnetometer manufactured by Riken Denshi Co., Ltd. The results indicated that both exhibit superparamagnetism, as shown in (a) and (b) of FIG. 8. This magnetism is specific to the aggregation of ferromagnetic fine particles, indicating that the properties of Fe₃O₄ nanoparticles are unchanged even after they are dispersed in glass beads. Since the vertical axis represents magnetization per unit mass, the magnetization of the Fe₃O₄ nanoparticles dispersed in the glass beads was smaller by the mass of the glass than that of the Fe₃O₄ nanoparticles alone. It was proved that the magnetization of the Fe₃O₄ nanoparticles alone at an applied magnetic field of 5 kOe was 37 emu/g, whereas the magnetization the glass beads in which the Fe₃O₄ nanoparticles were dispersed together with the CdTe nanoparticles, at an applied magnetic field of 5 kOe was 2.4 emu/g.

### Example 3

### Glass Beads 1 in which Different Types of Fluorescent Semiconductor Nanoparticles are Dispersed

Example 2 was slightly modified to produce green-emitting semiconductor nanoparticles, instead of the Fe₃O₄ nanoparticles exhibiting magnetism. Such semiconductor nanoparticles can be easily produced as CdTe nanoparticles (2.6 nm in diameter) by performing reflux for 30 minutes in Example 1, not for 100 hours. Thus, glass beads in which CdTe nanoparticles (2.6 nm in diameter) were dispersed in the portion where the Fe₃O₄ nanoparticles were dispersed in Example 2, were prepared.

Similarly, blue-emitting ZnSe-containing nanoparticles, which were synthesized in accordance with a document (Murase et al., Journal of Colloid and Interface Science, vol. 321, p. 468 (2008)), could be dispersed in the cavity portion of the glass beads, as in the green-emitting CdTe nanoparticles.

### Example 4

### Glass Beads 2 in which Different Types of Fluorescent Semiconductor Nanoparticles are Dispersed

Glass beads in which InP nanoparticles (III-V nanoparticles) were dispersed in the cavity portion were prepared as follows.

### (1) Production of InP nanoparticles

InP nanoparticles (III-V semiconductors) were produced by the solvothermal method in the following manner.

First, 0.4 g of indium chloride (InCl₃), 3 mL of trioctylphosphine ([CH₃(CH₂)₇]₃P, TOP), which is a surfactant, and 2.5 g of dodecylamine (CH₃(CF₂)₁₁NH₂, DDA) were added to an autoclave in a glove box under an argon gas atmosphere. Further, 5 mL of toluene (C₆H₅CH₃) was added as a solvent, and 0.5 mL of tris(dimethylamino)phosphine (C₆H₁₈N₃P) was added.

The autoclave was transferred to an electric furnace and maintained at 75° C. for 1 hour. Then, the temperature was further raised to 180° C., and the nanoparticles were allowed to grow for 24 hours. Toluene (10 mL) and 6 mL of methanol were added to the nanoparticle dispersion and sufficiently stirred. After centrifugation for 10 minutes, the transparent supernatant was removed to separate InP nanoparticles from by-products after the reaction.

Moreover, using the nanoparticle dispersion from which the by-products had been removed, nanoparticles with different particle diameters were taken using the size-selective precipitation method. Methanol was used as a poor solvent. When the solution became slightly turbid, it was centrifuged, and the resulting precipitate was taken and redispersed in hexane. The poor solvent was further added to the supernatant, and when the solution became turbid again thereby, it was centrifuged. This operation was repeated until no nanoparticles were present in the solution. Thus, the InP nanoparticles were divided into nanoparticles with narrow size distribution.

After the size selection, it was confirmed from the position and width of the first absorption peak that the particle diameters of the nanoparticles were sized. It was also confirmed from the results of X-ray diffraction that those produced and separated were InP nanoparticles. The PL efficiency at this time was as low as about 1%.

### (2) Production of Water-Dispersible InP Nanoparticles

The nanoparticles having different particle diameters, which were taken by the size-selective precipitation method in step (1), were dispersed in a mixture of butanol and hexane (the volume ratio of butanol to hexane is 1:2). At this time, the concentration of the nanoparticles in the solution was calculated from an absorption spectrum using the absorption coefficient of a previously published document (Adam et al., Journal of Chemical Physics, vol. 123, p. 084706 (2005)), and the result was about 0.3×10⁻⁵ mol/L. A small stirrer was put in a glass bottle and placed on a hot stirrer, and the temperature of the heater was set at 70° C. At this time, the temperature of the solution in the container was about 50° C.

While stirring the nanoparticle dispersion, 2 mL of a mixed solution containing zinc ion and thioglycolic acid (surfactant) (ZT solution) was added. In the ZT solution, the zinc ion concentration was 0.13 mol/L, and the molar ratio of zinc ion to thioglycolic acid was 1:2.43. Moreover, the pH value of the ZT solution was adjusted to 11.0 using sodium hydroxide.

After stirring, almost all the nanoparticles in the organic layer were dispersed in the aqueous layer, and the organic layer became completely transparent. On the other hand, the aqueous layer turned yellow or brown in color depending on the particle diameter of the nanoparticles. This aqueous solution was taken with a pipette and moved to a centrifuge tube. In order to remove the aggregated nanoparticles, centrifugation was carried out and then the supernatant was extracted. The nanoparticles after water dispersion slightly emitted PL.

When the supernatant was further put in a centrifuge tube and methanol was added as a poor solvent, the solution became turbid. This was centrifuged, thereby allowing the procurement of a powder of water-dispersible nanoparticles.

In the process of dispersing the InP nanoparticles in the aqueous solution, when no surfactant was present in the aqueous solution (ZT solution), the nanoparticles were not well dispersed in the aqueous solution and resulted in precipitates. Moreover, with no zinc ion, the nanoparticles were dissolved and disappeared by stirring the solution, and both the organic and aqueous layers became transparent.

### (3) Production of Semiconductor Nanoparticles Having a Core/Shell Structure

The powder of the water-dispersible nanoparticles obtained in step (2) was dissolved in the ZT solution described in step (2). The solution was irradiated with UV light in accordance with the method of a previously published document (Murase et al., Colloids and Surfaces A, vol. 294, p. 33 (2007)), thus forming zinc sulfide shells on the InP nanoparticles.

First, a small stirrer was put in the nanoparticle dispersion, and irradiated with UV light of a wavelength of 365 nm at an intensity of 4.0 W/cm² for 20 to 120 minutes, while stirring. The PL and absorption spectra were measured every 10 minutes.

The PL wavelength of the thus-prepared nanoparticles was 554 to 620 nm, and the PL efficiency was 34 to 47%. Table 1 shows the optical properties of the obtained nanoparticles.

Nanoparticles having a wide range of PL wavelengths between 450 to 750 nm could be obtained by adjusting the time for the solvothermal method in step (1).

TABLE 1
Properties of nanoparticles
after UV irradiation

| | Particle | PL | PL | |
|---|---|---|---|---|
| Sample | diameter | wavelength | efficiency | FWHM |
| No. | (nm) | (nm) | (%) | (nm) |
| 1 | 3.69 | 621 | 39.5 | 80.6 |
| 2 | 3.56 | 616 | 44.8 | 78.4 |
| 3 | 3.32 | 607 | 44.5 | 78.6 |
| 4 | 3.19 | 602 | 40.0 | 83.4 |
| 5 | 2.99 | 590 | 47.5 | 83.4 |
| 6 | 2.76 | 566 | 33.7 | 83.4 |
| 7 | 2.42 | 554 | 33.9 | 84.4 |

The InP nanoparticle-dispersed aqueous solution prepared in this way was used in place of the Fe₃O₄ nanoparticle-dispersed aqueous solution produced in Example 1 to produce glass beads in which the InP nanoparticles were dispersed in the cavity portion.

### Example 5

### Glass Beads in which Fluorescent Semiconductor Nanoparticles and Metal Nanoparticles are Dispersed

Gold nanoparticles were synthesized in accordance with a document (Nie et al., Journal of American Chemical Society, vol. 124, p. 9606 (2002)). The particle diameter was adjusted to 7 nm. After purification, ammonia was added to adjust the pH to 12 as in Example 1, thus preparing an aqueous solution (Y5).

As in Example 1, Igepal CO-520 (surfactant) was added to a cyclohexane solution and stirred, yielding a transparent solution. The nanoparticle dispersion (X5, pH 10; 2 mL) of Example 1 was added drop by drop to the transparent solution while vigorously stirring. Additionally, 0.1 mL of the aqueous solution (Y5) produced in this time was added drop by drop thereto and stirred for 24 hours. As a result, glass beads in which metal nanoparticles and semiconductor nanoparticles were dispersed were obtained.

### Example 6

### Glass Beads in which Metal Nanoparticles and Magnetic Nanoparticles are Dispersed

### (1) Production of Gold Nanoparticle Dispersion

The gold colloid solution prepared in Example 5 (2 mL, 1×10⁻⁵ mol/L, pH: 8.5), an aqueous ammonia solution (6.25 wt. %, 50 μL), and TEOS (20 μL) were mixed in a vial container. After the container was covered, the mixture was stirred for 2 hours, thus producing a dispersion (X6, pH 9) of gold nanoparticles coated with a thin silica layer.

### (2) Production of a Reverse Micellar Solution

A surfactant (Igepal CO-520; 3.52 g) was added to 25 g of cyclohexane, and stirred until the solution becomes transparent. This solution was taken in an amount of 2 mL and added dropwise to the above gold nanoparticle dispersion (X6) while vigorously stirring. The Fe₃O₄ nanoparticle (8 nm in particle diameter) dispersion (Y6, pH: 11) prepared in Example 2 was taken in an amount of 100 μL and added dropwise to the reverse micellar solution.

### (3) Production of Glass Beads

TEOS (150 μL) was added to the above reverse micellar solution and stirred for 24 hours to progress the reaction, thus preparing glass beads.

### Example 7

### Glass Beads in which a Drug and Semiconductor Nanoparticles are Dispersed

Ibuprofen (IBU), which is known as an anticancer drug, was purchased from Fluka. Referring to a recent known document (Advanced Functional Materials, vol. 18, p. 2780 (2008)), IBU was dispersed in an aqueous solution of polyvinyl alcohol, so that IBU was coated with polyvinyl alcohol. The dispersion was dispersed in an ammonia solution (pH 11) so that the amount was 10 wt. %. The InP nanoparticles produced by the method of Example 4 were dispersed in the glass phase by the method of Example 2. Simultaneously, IBU could be dispersed in the cavity portion of the glass sphere.

### Example 8

### Glass Beads on which an Antibody is Attached

1 μL of 2-[methoxy(polyethyleneoxy)propyl]trimethoxysilane (CH₃O(C₂H₄O)₆₋₉C₃H₆Si(OCH₃)₃) was added to the glass beads produced in Example 2, in which fluorescent semiconductor nanoparticles and magnetic nanoparticles were dispersed (0.3 mL in a 10 mmol/L PBS buffer solution) and stirred for 7 hours. Further, 0.2 mg of streptavidin-maleimide (purchased from Sigma) was added to a 10 mmol/L PBS buffer solution (20 μL) and stirred for 3 hours. Moreover, 100 μL of Biotin-SP-conjugated AffiniPure Goat Anti-Mouse IgG (H+L) (purchased from Jackson ImmunoResearch Laboratories, Inc.) was added thereto and stirred for 5 minutes. Thereafter, the sample was allowed to stand at 4° C. for 24 hours, so that IgG adhered to the glass beads. The glass beads were confirmed to emit PL by UV irradiation.

1. Silicon-containing glass beads having an average particle diameter of 20 nm to 1 μm, each having one or more cavities therein, the silicon-containing glass beads containing nanoparticles A in a glass phase of each of said silicon-containing glass beads.

2. The silicon-containing glass beads according to claim 1, further comprising a functional material in said one or more cavities.

3. The silicon-containing glass beads according to claim 2, wherein the functional material is nanoparticles B.

4. The silicon-containing glass beads according to claim 3, wherein said nanoparticles A and nanoparticles B each have an average particle diameter of 2 to 20 nm.

5. The silicon-containing glass beads according to claim 3, wherein said nanoparticles A and/or nanoparticles B are semiconductor nanoparticles.

6. The silicon-containing glass beads according to claim 3, wherein said nanoparticles A and/or nanoparticles B are semiconductor nanoparticles with a PL efficiency of not less than 20%.

7. The silicon-containing glass beads according to claim 5, wherein the semiconductor nanoparticles are at least one member selected from the group consisting of CdTe, CdSe, CdS, ZnSe, ZnSe₍₁₋ₓ₎Teₓ (0<x<1), ZnS, InP, InₓGa₍₁₋ₓ₎P (0<x<1), and InAs.

8. The silicon-containing glass beads according to claim 3, wherein the nanoparticles A and/or nanoparticles B are magnetic nanoparticles.

9. The silicon-containing glass beads according to claim 8, wherein the magnetic nanoparticles are at least one member selected from the group consisting of Fe₃O₄, Fe₂O₃, CoFe₂O₄, MnFe₂O₄, NiFe₂O₄, CoCrFeO₄, Pt, Co, PtCo, FePt, and FeCo.

10. The silicon-containing glass beads according to claim 8, which have a magnetization of 1 to 200 emu/g at an applied magnetic field of 5 kOe.

11. The silicon-containing glass beads according to claim 3, wherein said nanoparticles A and/or nanoparticles B are metal nanoparticles.

12. The silicon-containing glass beads according to claim 11, wherein the metal of the metal nanoparticles is at least one member selected from the group consisting of gold (Au), silver (Ag), and copper (Cu).

13. The silicon-containing glass beads according to claim 1, wherein the silicon-containing glass beads have cavities having an average inner diameter of 10 to 500 nm.

14. The silicon-containing glass beads according to claim 2, wherein the functional material is a pharmacologically active substance.

15. The silicon-containing glass beads according to claim 1, wherein an antibody is attached to the outer surface of each silicon-containing glass bead.

16. A method of producing silicon-containing glass beads having an average particle diameter of 20 nm to 1 μm, each having one or more cavities therein, the silicon-containing glass beads containing nanoparticles A in a glass phase of each of said silicon-containing glass beads), the method comprising the steps of:
(1) mixing a medium comprising a hydrophobic organic solvent and a surfactant with aqueous solution X comprising nanoparticles A and silicon alkoxide to prepare a reverse micellar solution; and
(2) adding alkaline aqueous solution Y to the reverse micellar solution prepared in step (1) to form the silicon-containing glass beads.

17. The method according to claim 16, wherein in step (2), said alkaline aqueous solution Y contains a functional material.

18. The method according to claim 16, wherein the hydrogen ion exponent of said silicon alkoxide aqueous solution X containing nanoparticles A (pH 1) of step (1) satisfies pH 1>7, and the hydrogen ion exponent of said alkaline aqueous solution Y (pH 2) of step (2) satisfies pH 1<pH 2<14.

19. The method according to claim 16, wherein step (2) comprising adding said alkaline aqueous solution Y to the reverse micellar solution prepared in step (1) and further adding thereto a silicon alkoxide to form the silicon-containing glass beads.

20. A fluorescence reagent comprising the silicon-containing glass beads according to claim 1.

21. A drug delivery system comprising the silicon-containing glass beads according to claim 1.
